(19) United States
(12) Patent Application Publication
(10) Pub. No.: US 2015/0320333 A1
(43) Pub. Date: Nov. 12, 2015

(54) Continuous Non-Ivasive Measurement of Tissue Temperatures Based on Impedance Measurements

(71) Applicant: Zimmer MedizinSysteme GmbH, Neu-Ulm (DE)

(72) Inventor: Armin Zimmer, Neu-Ulm (DE)

(21) Appl. No.: 14/802,433

(22) Filed: Jul. 17, 2015

Related U.S. Application Data
(63) Continuation of application No. PCT/EP2014/000084, filed on Jan. 15, 2014.
(60) Provisional application No. 61/755,626, filed on Jan. 23, 2013.

(30) Foreign Application Priority Data
Jan. 22, 2013 (DE) 1020130009669

(51) Int. Cl. A61B 5/053; A61B 5/01

(52) U.S. Cl. CPC A61B 5/053; A61B 5/01

(57) ABSTRACT

For the continuous, non-invasive measurement of temperatures in a tissue, a current is supplied to the tissue by means of at least one feed electrode. A voltage (U) caused by the current (I) is measured by means of at least one measuring electrode and from this the resistance or the magnitude of the impedance of the tissue through which the current flows is determined. The temperature in the tissue is determined directly from the resistance and/or the magnitude of the impedance.

### CROSS-REFERENCE TO RELATED APPLICATIONS

This application is a continuation-in-part of PCT/EP2014/000084, filed Jan. 15, 2014, which in turn claims priority to DE 10 2013 000 966.9, filed Jan. 22, 2013, and to U.S. Provisional Patent Application No. 61/755,626, filed Jan. 23, 2013. All of the foregoing applications are incorporated by reference herein in their entireties.

### BACKGROUND OF THE INVENTION

1. Field of Invention

The invention relates to a method for direct continuous non-invasive measurement of temperatures in a tissue, preferably at different depths as well as an apparatus, in particular for carrying out such a method.

2. Description of the Related Art

Hitherto in medicine, if one wished to measure the body internal temperature, this was usually accomplished invasively, i.e. using a measuring needle or a sensor inserted under the skin. A disadvantage of this method in addition to a certain risk of infection was that this was associated with not inconsiderable pain.

A medical measuring apparatus has become known from EP 2 096 989 B1 which determines the bioelectric impedance locally with electrodes. In EP 2 096 989 B1 the cardiac frequency and the pulse amplitude are determined with the aid of the bioelectric impedance. In turn, according to EP 2 096 989 B1 the body temperature can be determined from the pulse amplitude. A disadvantage with the measuring apparatus according to EP 2 096 989 B1 is that the body temperature cannot be derived directly but on the contrary a derivation from blood values is always necessary.

### BRIEF SUMMARY

It is therefore an object of the invention to provide a method and an apparatus which overcomes the disadvantages of the prior art and in particular enables the most exact possible determination of the temperature in simple and reproducible manner.

According to the invention, this object is solved by a method for the continuous non-invasive measurement of temperatures in a tissue, where a current is guided into the tissue by means of at least one feed electrode and a voltage caused by the current in the tissue is measured by means of at least one measuring electrode and from this the resistance or the impedance or the magnitude of the impedance of the tissue through which the current flows is determined. The invention is characterized in that a tissue temperature is determined directly from the measured resistance or the impedance or the magnitude of the impedance. The method described utilizes the fact that the resistance or the impedance or the magnitude of the impedance of the tissue increases or decreases with temperature. If a current is applied, the voltage correlates with the tissue temperature and the resistance or the impedance or the magnitude of the impedance determined from these quantities is a direct measure for the temperature prevailing in the tissue part.

For the continuous, non-invasive measurement of temperatures in a tissue, a current is supplied to the tissue by means of at least one feed electrode. A voltage (U) caused by the current (I) is measured by means of at least one measuring electrode and from this the resistance or the magnitude of the impedance of the tissue through which the current flows is determined. The temperature in the tissue is determined directly from the resistance and/or the magnitude of the impedance.

Example embodiments of the present general inventive concept can be achieved by a method for the continuous, non-invasive measurement of temperatures in a tissue, comprising: supplying a current to the tissue by means of at least one feed electrode and measuring a voltage caused by the current by means of at least one measuring electrode and from this the resistance or the magnitude of the impedance of the tissue through which the current flows is determined, characterized in that a tissue temperature in the tissue is determined directly from the resistance or the magnitude of the impedance.

Further example embodiments of the present general inventive concept can be achieved by a method characterized in that a reference temperature is determined by means of a measurement method and a certain resistance or a certain magnitude of the impedance is assigned.

Further example embodiments of the present general inventive concept can be achieved by a method characterized in that the measurement method performs the determination of the reference temperature with the aid of a sensor device, in particular a skin sensor and/or an IR thermometer.

Further example embodiments of the present general inventive concept can be achieved by a method characterized in that the method is carried out using at least two feed electrodes, a first feed electrode and a second feed electrode, wherein first and second feed electrode have a first distance from one another.

Further example embodiments of the present general inventive concept can be achieved by a method characterized in that the first distance between the first feed electrode and the second feed electrode is varied in order to determine the tissue temperature at various depths.

Further example embodiments of the present general inventive concept can be achieved by a method characterized in that the method is carried out using at least two measuring electrodes, a first measuring electrode and a second measuring electrode, wherein first and second measuring electrode have a second distance from one another.

Further example embodiments of the present general inventive concept can be achieved by a method characterized in that the first distance between the distance between the first measuring electrode and the second measuring electrode is varied in order to determine the tissue temperature at various depths.

Further example embodiments of the present general inventive concept can be achieved by a method characterized in that a current in a predefined frequency range is supplied by means of a frequency-variable generator and a frequency-dependent impedance is determined.

Further example embodiments of the present general inventive concept can be achieved by a method characterized in that the current is a frequency-variable alternating current, a pulsed direct current or a sinusoidal alternating current.

Further example embodiments of the present general inventive concept can be achieved by a method characterized in that the current is a frequency-variable alternating current and the frequency-variable alternating current is varied in its frequency over a frequency range.

Further example embodiments of the present general inventive concept can be achieved by a method characterized in that the frequency range is from a few Hz to several hundred MHz, in particular 10 kHz to 1000 kHz, preferably 300 kHz to 1000 kHz, preferentially 330 kHz to 900 kHz.

Example embodiments of the present general inventive concept can be achieved by an apparatus for continuous non-invasive measurement of temperatures in a tissue comprising: at least one feed electrode for feeding a current into a tissue; and at least one measuring electrode for measuring the voltage produced by the current in the tissue, characterized in that the apparatus comprises a unit for determining the resistance and/or the impedance and/or the magnitude of the impedance of the tissue through which current flows and the tissue temperature in the tissue directly from this.

Further example embodiments of the present general inventive concept can be achieved by an apparatus characterized in that the apparatus comprises a device for determining a reference temperature which is assigned a certain resistance or a certain magnitude of the impedance.

Further example embodiments of the present general inventive concept can be achieved by an apparatus characterized in that the device for determining the reference temperature is a sensor device, in particular a skin sensor and/or an IR thermometer.

Further example embodiments of the present general inventive concept can be achieved by an apparatus characterized in that the apparatus comprises a frequency-variable generator, in particular based on a microcontroller which provides a current in a predefined frequency range.

Further example embodiments of the present general inventive concept can be achieved by an apparatus characterized in that the frequency-variable generator is a tuneable generator.

Further example embodiments of the present general inventive concept can be achieved by an apparatus characterized in that the frequency-variable generator provides a monophase current or an alternating current having different signal

### BRIEF DESCRIPTION OF THE SEVERAL VIEWS OF THE DRAWINGS

The above-mentioned features and other aspects of the invention will become more clearly understood from the following detailed description of the invention read together with the drawings in which:

FIG. 1 a-1 b shows the measurement principle of the invention

FIG. 2 shows the Wenner electrode arrangement

FIG. 3a shows a family of characteristics for the apparatus according to the invention which shows the profile of the impedance value Z [Ω] for a measurement example to determine the temperature as a function of the frequency f [Hz] of the supplied alternating current for various temperatures.

FIG. 3b shows the temperature dependence of the impedance

FIG. 4 shows the structure of an apparatus according to the invention

### DETAILED DESCRIPTION

It is an object of the invention to provide a method and an apparatus which overcomes the disadvantages of the prior art and in particular enables the most exact possible determination of the temperature in simple and reproducible manner.

According to the invention, this object is solved by a method for the continuous non-invasive measurement of temperatures in a tissue, where a current is guided into the tissue by means of at least one feed electrode and a voltage caused by the current in the tissue is measured by means of at least one measuring electrode and from this the resistance or the impedance or the magnitude of the impedance of the tissue through which the current flows is determined. The invention is characterized in that a tissue temperature is determined directly from the measured resistance or the impedance or the magnitude of the impedance. The method described utilizes the fact that the resistance or the impedance or the magnitude of the impedance of the tissue increases or decreases with temperature. If a current is applied, the voltage correlates with the tissue temperature and the resistance or the impedance or the magnitude of the impedance determined from these quantities is a direct measure for the temperature prevailing in the tissue part.

The direct determination of the temperature of the tissue from the impedance measurement without a determination of blood values, as described for example, in EP 2 096 989 B1, has the advantage that even in tissue parts with poor circulation it is possible to determine the body temperature.

Preferably in the method according to the invention, in order to avoid galvanic effects an alternating current is fed as current into the tissue via the feed electrodes having a current density in the range of a few microamperes to a few milliamperes. The potential field formed in the tissue is then primarily dependent on the structure and temperature of the tissue.

The voltage formed in the tissue as a result of the potential field can be measured, for example using measuring electrodes. The specific resistance or the impedance or the magnitude of the impedance of the tissue through which current flows can then be calculated from the supplied current and the measured voltage taking into account the measurement geometry of the electrodes, where the specific resistance is the reciprocal of the specific conductance of the tissue.

For the specific resistance it holds that ρₛ=K*U/1, where U is the measured voltage, I is the supplied current and K is a geometrical factor which is dependent on the electrode arrangement of the measuring electrodes and the feed electrodes. The feed electrodes and the measuring electrodes can be provided in various arrangements, for example
a Wenner arrangement
a Schlumberger arrangement
a three-point system,
a double dipole system
a Lee arrangement

In the arrangement of feed and measuring electrodes according to the invention which preferably consists of a non-metallic material, a Wenner arrangement is preferred.

In order to determine the temperature or tissue temperature, a predefined frequency range, in particular from 330 kHz to 1000 kHz is covered with the aid of a phase locked loop (PLL), an impedance curve is recorded and at the same time as this, the variation of the resistance and resulting curve slopes are calculated.

Mathematical methods can also be used to be able to determine the tissue temperature.

In order to be able to determine the temperature in the tissue depth, in a further developed embodiment it can be provided to provide at least two feed electrodes and/or at least two measuring electrodes which have a distance from one another. If the distance is varied, the current penetrates to varying depth into the tissue. Values for the resistance, the impedance or the magnitude of the impedance can again be determined from the determined voltage for various distances. These values of the resistances, the impedances or the magnitude of the impedance can represent the temperatures in variously deep tissues.

If the absolute value for the temperature is to be determined from the determination of the resistance, the impedance or the magnitude of the impedance, it is advantageous to determine a reference temperature independently by means of an independent measurement method, e.g. a sensor device such as a skin sensor and/or an IR thermometer. A resistance determined by means of the method according to the invention, an impedance or a magnitude of an impedance or an impedance profile can be assigned to the independently determined reference temperature. Thus, a certain impedance value correlates with a certain temperature value. This enables the assignment of an absolute temperature.

Preferably an alternating current is supplied as current. However, a direct current or a pulsed direct current would also be possible. In order to be able to measure a frequency-dependent impedance, the applied current is frequency-variable.

By means of the reference temperature, the resistance, the impedance or the magnitude of the impedance or the impedance profile can be standardized or calibrated in relation to the temperature or a zero setting can be performed. If a frequency-variable current is supplied, the frequency range over which the current is varied and the impedance or the magnitude of the impedance is determined in a frequency-dependent manner is a range from a few Hz to several hundred MHz, in particular the range from 10 kHz to 1000 kHz, preferably from 300 kHz to 1000 kHz, quite preferably from 330 kHz to 900 kHz.

In addition to the method, the invention also provides an apparatus for noninvasive measurement of the tissue temperature. The apparatus according to the invention comprises at least one feed electrode for supplying a current into a tissue and at least one measuring electrode. The feed electrodes can consist of metallic or also of non-metallic materials, for example, it would be possible to make the feed electrodes from stainless steel. In addition to the feed electrodes for supplying the current, the apparatus comprises at least one measuring electrode which can also be fabricated from a metallic or non-metallic material. The measuring electrodes can be AgAgCI multiple electrodes in the form, for example, of Ag—AgCI plates or Ag—AgCI single electrodes preferably as adhesive electrodes. The AgAgCI electrodes are not suitable as feed electrodes as a result of their chemical properties. In addition to the feed electrodes and the measuring electrodes, the apparatus further comprises a unit with the aid of which it is possible to determine the resistance or the impedance or the magnitude of the impedance or the impedance profile in the tissue through which current flows from the supplied current and the measured voltage and then determine the temperature in the tissue from the determined resistance or the determined impedances or the determined impedance profile over the frequency. To this end, various impedance values are determined which are measured by frequency variability relating to their impedance.

It is particularly preferred if the apparatus according to the invention comprises a frequency-variable generator by which means the current is supplied. A possible generator would be a tuneable sine generator having a frequency range of 300 kHz to 1000 kHz, which delivers constant current in the range of a few μA to a few mA. The recording of the measured values is then made in the mV range, i.e. at a voltage of <100 mV. It is particularly preferable if the frequency-variable generator is based on a microcontroller circuit. In one embodiment of the invention it can be provided that the tuneable generator is in particular a generator which is tuneable by means of a phase-locked loop (PLL).

In addition to a sine signal having one frequency, other periodic signals are also feasible such as, for example, a rectangular or triangular signal which can also be varied in their frequency. Curve shapes other than sine signals for the feed signal have the advantage that other harmonics can be specifically set. In addition to periodic signals, monophase current pulses are also possible.

As described previously, a sensor device can be provided for zero setting or calibration. With the aid of the sensor device it is possible to determine an absolute temperature independently of the impedance measurement, e.g. the surface temperature of the skin. This absolute value can then again be assigned an impedance which is present at the absolute temperature so that impedance values correlated with the temperature. Since a non-invasive method is involved here, surface sensors which can be applied to the skin are provided for this purpose.

The invention will be described in more detail hereinafter with reference to exemplary embodiments.

In the figures:

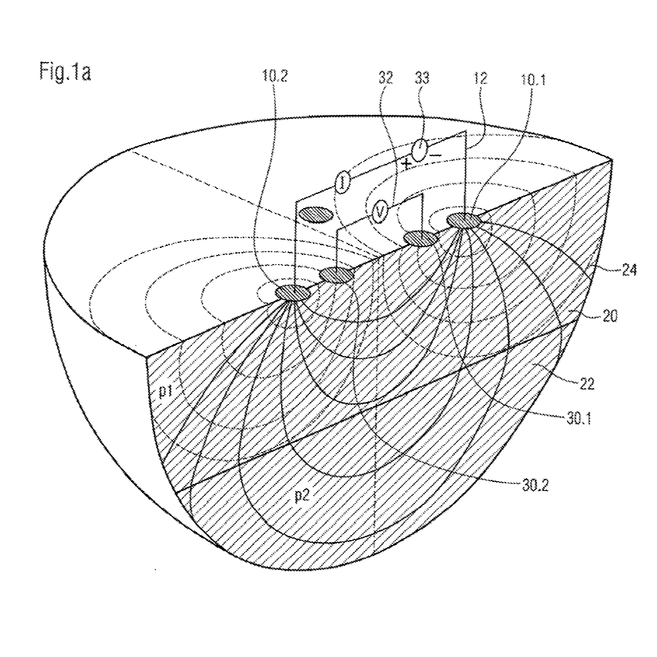

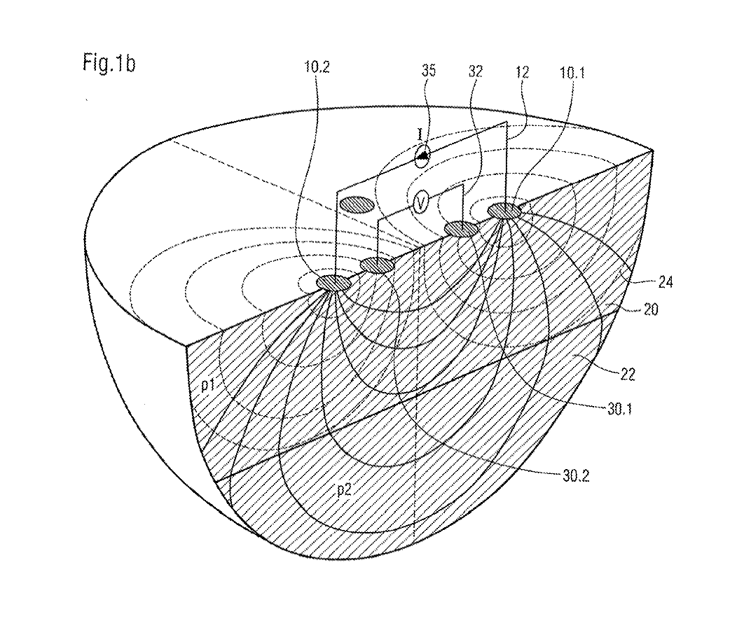

FIG. 1 a-1 b shows the measurement principle of the invention

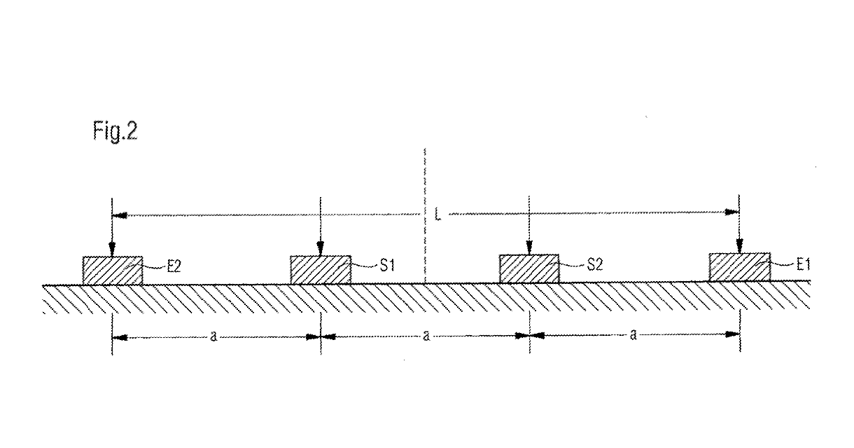

FIG. 2 shows the Wenner electrode arrangement

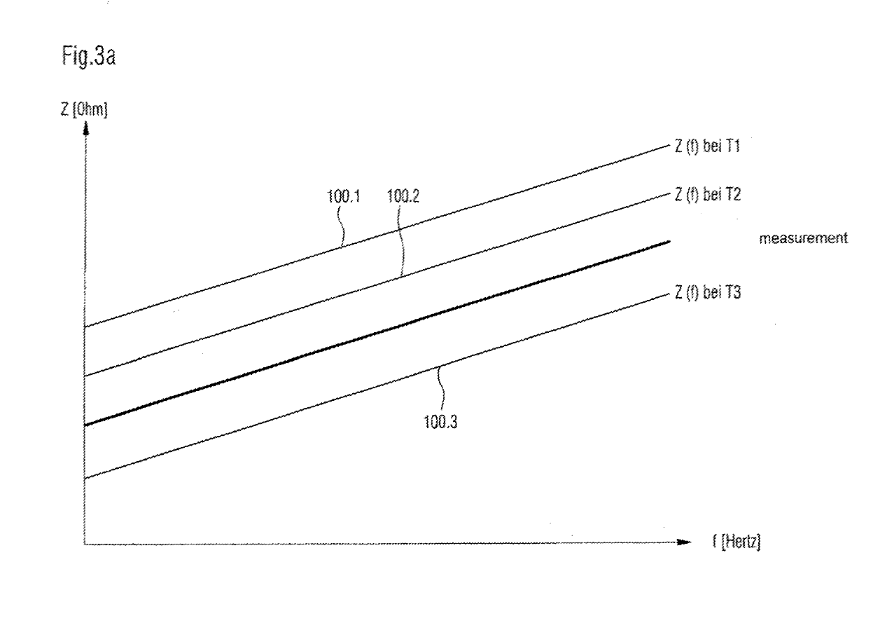

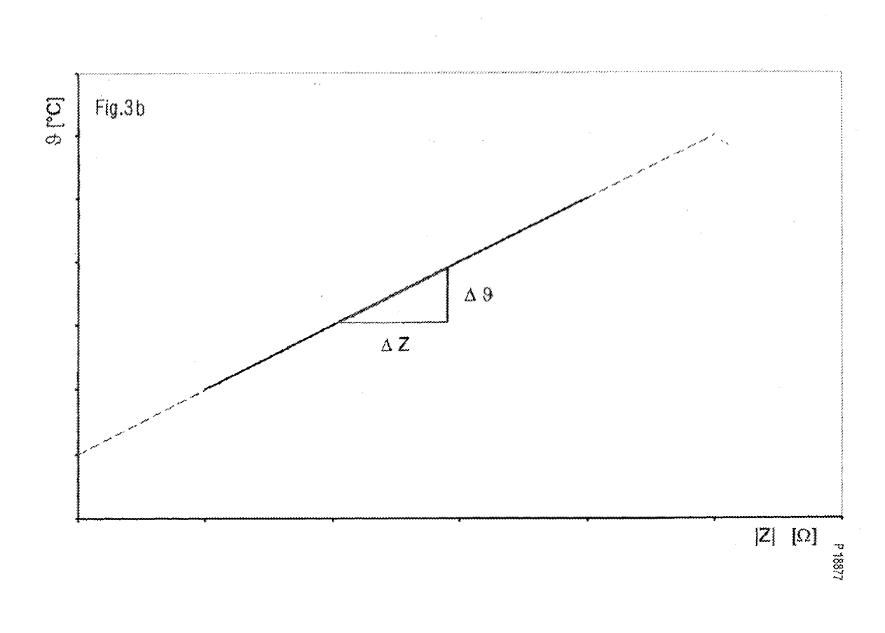

FIG. 3a shows a family of characteristics for the apparatus according to the invention which shows the profile of the impedance value Z [Ω] for a measurement example to determine the temperature as a function of the frequency f [Hz] of the supplied alternating current for various temperatures.

FIG. 3b shows the temperature dependence of the impedance

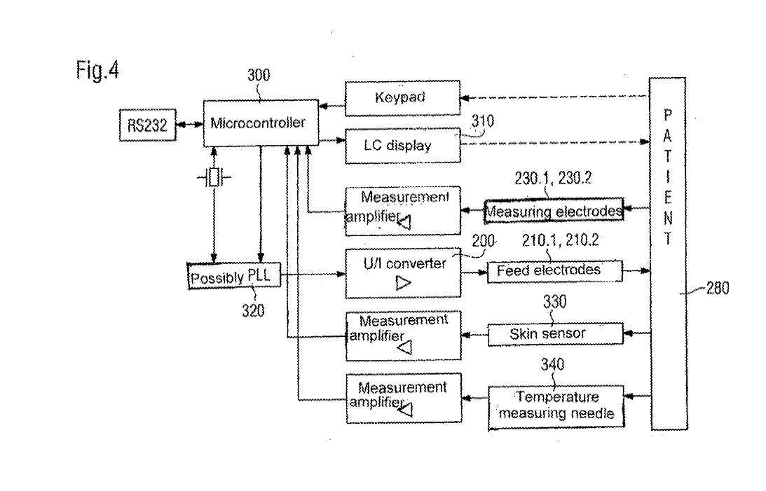

FIG. 4 shows the structure of an apparatus according to the invention

FIGS. 1a and 1b show the measurement principle of the method. In the method according to the invention, in the embodiment being considered, the electrodes are, without being restricted to this, as shown in FIG. 2, arranged according to Wenner, i.e. two feed electrodes 10.1, 10.2 are provided by which means a current 12 is fed into a tissue 20 lying below the electrodes. The current lines caused by the current inside the tissue are characterized by reference number 22. As a result of the current flow through the tissue 20, a potential field with potential lines 24 is formed and a voltage 32 is determined with the aid of the measuring electrodes 30.1, 30.2. From the measured voltage, knowing the supplied current, the resistance or the impedance or the magnitude of the impedance or an impedance value can be determined which is a direct measure for the temperature in the tissue, as shown hereinafter. FIG. 1a shows the supply of current with the aid of a voltage source 33.

Alternatively FIG. 1 b shows the supply with a current source 35. The same components as in FIG. 1a are characterized by the same reference numbers. In the method according to the invention in a special embodiment, FIG. 2 shows a special Wenner electrode arrangement—without being restricted to this. In the Wenner electrode arrangement, the feed electrodes E1, E2 should be equated to the electrodes 10.1, 10.2 shown in FIG. 1a, the measuring electrodes S1, S2 are designated by 30.1 and 30.2 in FIG. 1a. The distance between the feed electrodes E1, E2 is specified by L, the distance between feed electrode E1 and measuring electrode S1 as well as measuring electrodes S1 and S2 and measuring electrode S2 and feed electrode E2 is always equidistant as a. The geometric factor K for the Wenner arrangement is then obtained as K=a. Although a Wenner electrode arrangement is shown here, other electrode arrangements are also feasible, such as a Schlumberger arrangement, a three-point system, a double dipole system or a Lee arrangement. The electrodes substantially differ by the electrode placement and the geometric factor K.

FIG. 3a shows a family of characteristics plotted for the apparatus according to the invention which shows the behaviour of the impedance value Z [Ω] as a function of the frequency f [Hz] of the supplied alternating current for various temperatures. As can be seen from FIG. 3, for each temperature T1, T2, T3, a specific characteristic 100.1, 100.2, 100.3 is determined as a function of the frequency for the impedance. As shown in FIG. 3, a family of characteristics of the frequency-dependent impedance Z[Ω] is thus obtained depending on the body temperature T in Kelvin. The behaviour Z [Ω] of the tissue impedance in the frequency range between 330 kHz and 950 kHz in the exemplary embodiment shown without being restricted to this is linear with respect to the tissue temperature to be measured, i.e. the lines for the temperatures T1, T2 and T3 are displaced parallel to one another and the distance of the lines for different temperatures is equidistant for different frequencies. In the specified frequency range of 330 kHz to 950 kHz, as shown in FIG. 3b, this has the result that a linear relationship is found between temperature and impedance in the specified frequency range. FIG. 3b shows this linear relationship. Naturally as a result of the equidistance of the curves in FIG. 3a the linear relationship between temperature and impedance also applies when the impedance is determined for an averaged frequency in the specified frequency range. The determination of an impedance over an averaged frequency range e.g. from 330 kHz to 950 kHz is advantageous since a subsequent averaging will improve the result in most cases. If the body temperature at the observed location changes, e.g. due to supply of heat, for example, during a heat treatment of the tissue, the tissue temperature and thus the impedance for a certain frequency or a certain frequency range or an averaged frequency increase. As a result of the linearity of impedance Z[Ω] and temperature d [° C.], as shown in FIG. 3b, the temperature rise AO can be determined from the increase in impedance ΔZ.

As a result of this behaviour, it is possible to carry out non-invasive temperature measurements by the indirect route of the tissue impedance in the range of 330 kHz to 950 kHz. The family of characteristics is determined whereby, for example, a tissue surface or a skin sample is heated to different temperatures by means of a heating device, for example, a heat lamp. In order to be able to assign absolute temperatures to the impedance values, reference measurements can be carried out. A possible reference temperature can be the surface temperature or the ear temperature of the patient. The surface temperature can be measured, for example, with the aid of a surface sensor as reference.

If a special measurement is carried out, knowing the family of characteristics as described for FIG. 3a and FIG. 3b, the temperature can be determined. The straight line shown in FIG. 3b which gives the relationship of impedance and temperature is a direct measure for the temperature. The variable frequency substantially increases the measurement accuracy of the method since an averaged impedance value for a frequency range can be assigned to a temperature value. This averaged impedance value also varies linearly with temperature so that a temperature rise in the tissue can be detected directly through an increase in the impedance value. It is particularly preferred if the parameter field shown in FIG. 3a is a standardized Z-f parameter field which can be used for all measurements. The determination of temperature is then confined to reading off values.

A schematic diagram of the apparatus for determining the tissue temperature is described in FIG. 4.

The apparatus according to the invention comprises on the one hand a U-1 converter 200, by which means a current, preferably a constant current, is applied to the tissue of a patient 280 via the feed electrodes 210.1, 210.2. The current acting on the tissue of the patient 280 leads to the formation of a potential field and thus to a voltage which can be determined by means of the measuring electrodes 230.1, 230.2. The voltage received by the measuring electrodes 230.1, 230.2 is amplified by the measuring amplifier and supplied to a microcontroller 300. In the microcontroller the frequency-dependent impedance is evaluated and calculated from the applied current and the measured voltage. The impedance is in turn displayed on an LCD display 310 as a function of the frequency. The microcontroller 300 further controls the frequency-variable generator 320, which can be configured as a PLL generator and whose signal is converted via the U-1 converter 200 into a current with constant amplitude which is supplied to the patient via the feed electrodes 210.1, 210.2. For standardization and calibration purposes it can be provided that, in addition to the electronic and computational determination of the tissue temperature by means of the measured voltage and the supplied current intensity, a reference measurement is made, for example, with the aid of a skin sensor 330 or a temperature measuring needle 340, which can record a depth-dependent temperature in the tissue. The skin sensor 330 is a special type of surface sensor whereas the temperature measuring needle enables a depth-dependent measurement. Both measurement methods can be used for calibration and/or standardization purposes.

With the apparatus according to the invention, an apparatus and a method are provided for the first time which allow the body temperature of a proband to be determined non-invasively directly in a very simple manner by means of a simple impedance measurement. The method and the apparatus are suitable for all areas of temperature acquisition such as long-term recordings or bedside monitoring or monitoring. Furthermore it can be used in intensive care, in operating and anaesthesia operation and in tumour therapy and in particular for monitoring temperature in therapies or applications in which heat or cold is applied to patients.

Example embodiments of the present general inventive concept can be achieved by a method for the continuous, non-invasive measurement of temperatures in a tissue, wherein a current is supplied to the tissue (20) by means of at least one feed electrode (0.1, 10.2) and a voltage (U) caused by the current (I) is measured by means of at least one measuring electrode (30.1, 30.2) and from this the resistance or the magnitude of the impedance of the tissue (20) through which the current flows is determined, characterized in that a tissue temperature in the tissue is determined directly from the resistance and/or the magnitude of the impedance.

Further example embodiments of the present general inventive concept can be achieved by a method as described above, characterized in that a reference temperature is determined by means of a measurement method and a certain resistance or a certain magnitude of the impedance is assigned.

Further example embodiments of the present general inventive concept can be achieved by any of the methods as described above, characterized in that the measurement method performs the determination of the reference temperature with the aid of a sensor device, in particular a skin sensor and/or an IR thermometer.

Further example embodiments of the present general inventive concept can be achieved by any of the methods as described above, characterized in that the method is carried out using at least two feed electrodes, a first feed electrode and a second feed electrode, wherein first and second feed electrode have a first distance from one another.

Further example embodiments of the present general inventive concept can be achieved by any of the methods as described above, characterized in that the method is carried out using at least two measuring electrodes, a first measuring electrode and a second measuring electrode, wherein first and second measuring electrode have a second distance from one another.

Further example embodiments of the present general inventive concept can be achieved by any of the methods as described above, characterized in that the first distance between the first and second feed electrode and/or the distance between the first and second measuring electrode is varied in order to determine the tissue temperature at various depths.

Further example embodiments of the present general inventive concept can be achieved by any of the methods as described above, characterized in that a current in a predefined frequency range is supplied by means of a frequency-variable generator and the frequency-dependent impedance is determined.

Further example embodiments of the present general inventive concept can be achieved by any of the methods as described above, characterized in that the current is a frequency-variable alternating current, a pulsed direct current or a sinusoidal alternating current.

Further example embodiments of the present general inventive concept can be achieved by any of the methods as described above, characterized in that the frequency-variable alternating current is varied in its frequency over a frequency range.

Further example embodiments of the present general inventive concept can be achieved by any of the methods as described above, characterized in that the frequency range is from a few Hz to several hundred MHz, in particular 10 kHz to 1000 kHz, preferably 300 kHz to 1000 kHz, preferentially 330 kHz to 900 kHz.

Example embodiments of the present general inventive concept can be achieved by an apparatus for continuous non-invasive measurement of temperatures in a tissue (20) comprising at least one feed electrode (10.1, 0.2) for feeding a current into a tissue; at least one measuring electrode (30.1, 30.2) for measuring the voltage produced by the current in the tissue, characterized in that the apparatus comprises a unit for determining the resistance and/or the impedance and/or the magnitude of the impedance of the tissue (20) through which current flows and the tissue temperature in the tissue directly from this.

Further example embodiments of the present general inventive concept can be achieved by the apparatus as described above, characterized in that the apparatus comprises a device for determining a reference temperature which is assigned a certain resistance or a certain magnitude of the impedance.

Further example embodiments of the present general inventive concept can be achieved by any of the apparatuses as described above, characterized in that the device for determining the reference temperature is a sensor device, in particular a skin sensor and/or an IR thermometer.

Further example embodiments of the present general inventive concept can be achieved by any of the apparatuses as described above, characterized in that the apparatus comprises a frequency-variable generator, in particular based on a microcontroller which provides a current in a predefined frequency range.

Further example embodiments of the present general inventive concept can be achieved by any of the apparatuses as described above, characterized in that the frequency-variable generator is a tuneable generator.

Further example embodiments of the present general inventive concept can be achieved by any of the apparatuses as described above, characterized in that the frequency-variable generator provides a monophase current or an alternating current having different signal shapes, in particular a rectangular shape, a triangular shape or a sine shape

Further example embodiments of the present general inventive concept can be achieved by use of a method according to the foregoing or an apparatus according to the foregoing for at least one of the following purposes: for long-term recording; for monitoring or bed-side monitoring; for intensive care, particular in operating and anaesthesia operation and tumour therapy; for monitoring the temperature or temperature behaviour, in particular in therapy or applications in which heat or cold is applied to the patient.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

### Examples

By way of example, and not by way of limitation, the present general inventive concept comprises aspects which are described in the following sentences and constitute a part of the description of the present general inventive concept:

1. Method for the continuous, non-invasive measurement of temperatures in a tissue, wherein a current is supplied to the tissue (20) by means of at least one feed electrode (10.1, 10.2) and a voltage (U) caused by the current (I) is measured by means of at least one measuring electrode (30.1, 30.2) and from this the resistance or the magnitude of the impedance of the tissue (20) through which the current flows is determined, characterized in that a tissue temperature in the tissue is determined directly from the resistance and/or the magnitude of the impedance.

2. The method according to sentence 1, characterized in that a reference temperature is determined.

3. The method according to one of sentences 1 to 2, characterized in that the reference temperature is with the aid of a sensor device, in particular a skin sensor and/or an IR thermometer.

4. The method according to one of sentences 1 to 3, characterized in that the method is carried out using at least two feed electrodes, a first feed electrode and a second feed electrode, wherein first and second feed electrode have a first distance from one another.

5. The method according to one of sentences 1 to 4, characterized in that the method is carried out using at least two measuring electrodes, a first measuring electrode and a second measuring electrode, wherein first and second measuring electrode have a second distance from one another.

6. The method according to sentence 4, characterized in that the first distance between the first and second feed electrode and/or the distance between the first and second measuring electrode is varied in order to determine the tissue temperature at various depths.

7. The method according to one of sentences 1 to 6, characterized in that a predefined frequency range is covered by means of a frequency-variable generator and the frequency-dependent impedance is determined.

8. The method according to one of sentences 1 to 7, characterized in that the current is a frequency-variable alternating current, a pulsed direct current or a sinusoidal alternating current.

9. The method according to one of sentences 1 to 8, characterized in that the frequency-variable alternating current is varied in its frequency over a frequency range.

10. The method according to sentence 9, characterized in that the frequency range is from a few Hz to several hundred MHz, in particular 10 kHz to 1000 kHz, preferably 300 kHz to 1000 kHz, preferentially 330 kHz to 900 kHz.

11. Apparatus for continuous non-invasive measurement of temperatures in a tissue (20) comprising at least one feed electrode (10.1, 10.2) for feeding a current into a tissue; at least one measuring electrode (30.1, 30.2) for measuring the voltage produced by the current in the tissue, characterized in that the apparatus comprises a unit for determining the resistance and/or the impedance and/or the magnitude of the impedance of the tissue (20) through which current flows.

12. The apparatus according to sentence 11, characterized in that the apparatus comprises a device for determining a reference temperature.

13. The apparatus according to sentence 12, characterized in that the device for determining the reference temperature is a sensor device, in particular a skin sensor and/or an IR thermometer.

14. The apparatus according to one of sentences 11 to 13, characterized in that the apparatus comprises a frequency-variable generator, in particular based on a microcontroller which provides a current in a frequency-dependent manner.

15. The apparatus according to sentence 14, characterized in that the frequency-variable generator is a tuneable generator, in particular a generator tuneable by means of a phase-locked loop (PLL).

16. The apparatus according to one of sentences 14 to 15, characterized in that the frequency-variable generator provides a monophase current or an alternating current having different signal shapes, in particular a rectangular shape, a triangular shape or a sine shape.

17. The apparatus according to sentence 15, characterized in that the monophase current or the alternating current is provided with constant or variable amplitude.

18. Use of a method according to one of sentences 1 to 10 or an apparatus according to one of sentences 11 to 17 for at least one of the following purposes:
for long-term recording
for monitoring or bed-side monitoring
for intensive care, particular in operating and anaesthesia operation and tumour therapy
for monitoring the temperature or temperature behaviour, in particular in therapy or applications in which heat or cold is applied to the patient.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. A method for the continuous, non-invasive measurement of temperatures in a tissue, comprising:
supplying a current to the tissue by means of at least one feed electrode and
measuring a voltage caused by the current by means of at least one measuring electrode and from this the resistance or the magnitude of the impedance of the tissue through which the current flows is determined, characterized in that a tissue temperature in the tissue is determined directly from the resistance or the magnitude of the impedance.

2. The method according to claim 1, characterized in that a reference temperature is determined by means of a measurement method and a certain resistance or a certain magnitude of the impedance is assigned.

3. The method according to claim 2, characterized in that the measurement method performs the determination of the reference temperature with the aid of a sensor device, in particular a skin sensor and/or an IR thermometer.

4. The method according to claim 1, characterized in that the method is carried out using at least two feed electrodes, a first feed electrode and a second feed electrode, wherein first and second feed electrode have a first distance from one another.

5. The method according to claim 4, characterized in that the first distance between the first feed electrode and the second feed electrode is varied in order to determine the tissue temperature at various depths.

6. The method according to claim 1, characterized in that the method is carried out using at least two measuring electrodes, a first measuring electrode and a second measuring electrode, wherein first and second measuring electrode have a second distance from one another.

7. The method according to claim 6, characterized in that the first distance between the distance between the first measuring electrode and the second measuring electrode is varied in order to determine the tissue temperature at various depths.

8. The method according to claim 1, characterized in that a current in a predefined frequency range is supplied by means of a frequency-variable generator and a frequency-dependent impedance is determined.

9. The method according to claim 1, characterized in that the current is a frequency-variable alternating current, a pulsed direct current or a sinusoidal alternating current.

10. The method according to claim 9, characterized in that the current is a frequency-variable alternating current and the frequency-variable alternating current is varied in its frequency over a frequency range.

11. The method according to claim 10, characterized in that the frequency range is from a few Hz to several hundred MHz, in particular 10 kHz to 1000 kHz, preferably 300 kHz to 1000 kHz, preferentially 330 kHz to 900 kHz.

12. An apparatus for continuous non-invasive measurement of temperatures in a tissue comprising:
at least one feed electrode for feeding a current into a tissue; and
at least one measuring electrode for measuring the voltage produced by the current in the tissue, characterized in that the apparatus comprises a unit for determining the resistance and/or the impedance and/or the magnitude of the impedance of the tissue through which current flows and the tissue temperature in the tissue directly from this.

13. The apparatus according to claim 12, characterized in that the apparatus comprises a device for determining a reference temperature which is assigned a certain resistance or a certain magnitude of the impedance.

14. The apparatus according to claim 13, characterized in that the device for determining the reference temperature is a sensor device, in particular a skin sensor and/or an IR thermometer.

15. The apparatus according to claim 12, characterized in that the apparatus comprises a frequency-variable generator, in particular based on a microcontroller which provides a current in a predefined frequency range.

16. The apparatus according to claim 15, characterized in that the frequency-variable generator is a tuneable generator.

17. The apparatus according to claim 15, characterized in that the frequency-variable generator provides a monophase current or an alternating current having different signal
